SEQUENCE LISTING (36 sequences)

SEQ ID NO: 1 - DNA, 2490 bp, Homo sapiens
  CDS (73)..(1122)
agaggatccgggttcggttgctctggcgagggctccagcatcacaggcggcggctgcgggcgcagagcggagatgcagcggcttggggccaccctgctgtgcctgctgctggcggcggcggtccccacggcccccgcgcccgctccgacggcgacctcggctccagtcaagcccggcccggctctcagctacccgcaggaggaggccaccctcaatgagatgttccgcgaggttgaggaactgatggaggacacgcagcacaaattgcgcagcgcggtggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgggggagcctgcggctgccgccgctgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttcccctctggcttgacagcatgaggtgttgtgcatttgttcagctcccccaggctgttctccaggcttcacagtctggtgcttgggagagtcaggcagggttaaactgcaggagcagtttgccacccctgtccagattattggctgctttgcctctaccagttggcagacagccgtttgttctacatggctttgataattgtttgaggggaggagatggaaacaatgtggagtctccctctgattggttttggggaaatgtggagaagagtgccctgctttgcaaacatcaacctggcaaaaatgcaacaaatgaattttccacgcagttctttccatgggcataggtaagctgtgccttcagctgttgcagatgaaatgttctgttcaccctgcattacatgtgtttattcatccagcagtgttgctcagctcctacctctgtgccagggcagcattttcatatccaagatcaattccctctctcagcacagcctggggagggggtcattgttctcctcgtccatcagggatctcagaggctcagagactgcaagctgcttgcccaagtcacacagctagtgaagaccagagcagtttcatctggttgtgactctaagctcagtgctctctccactaccccacaccagccttggtgccaccaaaagtgctccccaaaaggaaggagaatgggatttttcttttgaggcatgcacatctggaattaaggtcaaactaattctcacatccctctaaaagtaaactactgttaggaacagcagtgttctcacagtgtggggcagccgtccttctaatgaagacaatgatattgacactgtccctctttggcagttgcattagtaactttgaaaggtatatgactgagcgtagcatacaggttaacctgcagaaacagtacttaggtaattgtagggcgaggattataaatgaaatttgcaaaatcacttagcagcaactgaagacaattatcaaccacgtggagaaaatcaaaccgagcagtgctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaattatgtataaacataagttgcatttagaaatcaagcataaatcacttcaactgctaaaaaaa

SEQ ID NO: 2 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALGEPAAAAAALLGGEEI

SEQ ID NO: 3 - protein, 344 aa, Gallus sp.
PAPRRRWLLLLAVLAALCCAAAGSGGRRRAASLGEMLREVEALMEDTQHKLRNAVQEMEAEEEGAKKLSEVNFENLPPTYHNESNTETRIGNKTVQTHQEIDKVTDNRTGSTIFSETIITSIKGGENKRNHECIIDEDCETGKYCQFSTFEYKCQPCKTQHTHCSRDVECCGDQLCVWGECRKATSRGENGTICENQHDCNPGTCCAFQKELLFPVCTPLPEEGEPCHDPSNRLLNLITWELEPDGVLERCPCASGLICQPQSSHSTTSVCELSSNETRKNEKEDPLNMDEMPFISLIPRDILSDYEESSVIQEVRKELESLEDQAGVKSEHDPAHDLFLGDEI

SEQ ID NO: 4 - DNA, 28 bp, Artificial Sequence
  Oligonucleotide primer
gggaggatccgcgcccgctccgacggcg

SEQ ID NO: 5 - DNA, 34 bp, Artificial Sequence
  Oligonucleotide primer
gcctctagattaaatctcttcccctcccagcagt

SEQ ID NO: 6 - DNA, 38 bp, Artificial Sequence
  Oligonucleotide primer
tgccgcggatccgccatcatgcagcggcttggggccac

SEQ ID NO: 7 - DNA, 49 bp, Artificial Sequence
  Oligonucleotide primer
gcacaggtacccacagcctggtccagatctaaatctcttcccctcccag

SEQ ID NO: 8 - DNA, 35 bp, Artificial Sequence
  Oligonucleotide primer
gtctctagacagatctaaatctcttcccctcccag

SEQ ID NO: 9 - DNA, 65 bp, Artificial Sequence
  Oligonucleotide primer
gtctctagacagatctaagcgtagtctgggacgtcgtatgggtaaatctcttcccctcccagcag

SEQ ID NO: 10 - DNA, 41 bp, Artificial Sequence
  Oligonucleotide primer
gctgccgcggatccgccaccatgcagcggcttggggccacc

SEQ ID NO: 11 - DNA, 34 bp, Artificial Sequence
  Oligonucleotide primer
cacacgcggatccagatctaaatctcttcccctc

SEQ ID NO: 12 - DNA, 384 bp, Homo sapiens
  misc_feature (45)..(45): May be any nucleotide
aattcggcacgagcctgactgaagagatggcgctgagggagcctncggctgccgccgttgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttccccnctggcttgacagcatgaggtgttgtgcattttgttcagctcccccaggctgttctccaggnttcacagtctggtgcttgggagagtnaaggcagggttaaacttcaggagcagtttgccacccntngtncngattatttggcttgctttnccnntaccatttgcaaaanagccgttt

SEQ ID NO: 13 - DNA, 503 bp, Homo sapiens
  misc_feature (262)..(262): May be any nucleotide
aattcggcacgaggttccgcgaggttgaggaactgatggaggacacgcagcacaaattgcgcacgcggtggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagacttgacaaatggtctttttcagagacagtttnacatctttgggagacgagaagcagaggngcncgntncntatngcgnggcttttggccagattnctnccatttncagttccntaactncacctnccggccanggtntttacccggcatngtttttggccactttntttggtatnnccaatgcccnggagatngcctttnnacanggntcaccggttttttnttcaagggttttcttttaaatcctggggggttcttcccacgtttgntttct

SEQ ID NO: 14 - DNA, 490 bp, Homo sapiens
  misc_feature (184)..(184): May be any nucleotide
ggcacgagggaggcttgaggtggaagtgggggtcgggcactctgacctggtcgaggaggggctagggtttgaaccggggacagagtctaggtgagctggggcttgggagctattagcgtagaggatccgggttcggttgctctggcgagggctccagcatcacaggcggcggctgcgggcgcanagcgtagatgcagcggcttggggccaccctgctgtgcctgctgctggcggcggcggtccccacggcccccgcgcccgctccgacggcgacctcggctccagtcaagcccggcccggctctggactnacccgcagagggangccaacctncaatggaaatgtttccgcgnagtttggaggaatngatgggaaggacacgcnannanaaattgcgcnagcggttgggaagagatggaaggcaagaaagaagttgctggtgaaagnatcatcaagaaatggaacttggcaaattgaactccccannant

SEQ ID NO: 15 - DNA, 84 bp, Homo sapiens
  misc_feature (32)..(32): May be any nucleotide
ggcgacctcggctccagtcaagcccggcccgngntctcagctacccataggtggaggncaccctgngtgcanacccttgcccaa

SEQ ID NO: 16 - DNA, 221 bp, Homo sapiens
  misc_feature (30)..(30): May be any nucleotide
caatgtggagtctccctctgattggttttngggaaatgtggagaagagtgccctgctttgcaaacatcaacctggcaaaaatgcaacaaatgnattttccacgcattctttccatgggcataggtaagctgtgccttcagctgttgcagatgaaatgttctgttcaccctgcattacatgtgtttattcatccagcagtgttgctcagctcctacctctgt

SEQ ID NO: 17 - DNA, 557 bp, Homo sapiens
  misc_feature (283)..(283): May be any nucleotide
gccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggaggngagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatctgctgcccagagaggtcccgatgagtatgaagttggaacttcatggaggaggtncgcaagaacttgaagacttgagaggagcttactgaanaat

SEQ ID NO: 18 - DNA, 410 bp, Homo sapiens
  misc_feature (379)..(379): May be any nucleotide
gggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctgggagaggagccttgacttnaagagatggcgctgagggagccttcgggttg

SEQ ID NO: 19 - DNA, 397 bp, Homo sapiens
  misc_feature (1)..(1): May be any nucleotide
naagttggcagcttcatggaggaggtgcgccaggagctggaggacctggagaggagcctgactgaagagatggcgctgggggagcctgcgctgccgccttggcantgctgggaggggaagagatttagatctggaccaggctgtgggtagatgtgcaatagaaatagctaatttatttccccaggtntgtgctttaggcgtgggctgaccaggcttcttccnacatcttcttcccagtaagtttcccctctggcttgacagcatgaggtgttntgcatttgttcagctcccccaggctgttctccaggcttcacagtcttgtgcttgggagagtcaggcagggttaaactgcaggagcagtttgccacccctgtccagattatttggctgctttgcc

SEQ ID NO: 20 - DNA, 356 bp, Homo sapiens
  misc_feature (203)..(203): May be any nucleotide
tgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactnccagccggggctgtgctgtgccttccagagaggcctgctgttccctgtgtgcacanccctgcccgtggagggcgagctttgccatgaccccgncagccggnttctggacctcatcaactgggagctagagcctgatggagccttggaccgatg

SEQ ID NO: 21 - DNA, 319 bp, Homo sapiens
  misc_feature (5)..(5): May be any nucleotide
ctggngaggagcctgactgaagagatggcgctgggggagcctgcggctgccgccgctgcactgctgggaggggaagagatttagatctggaccaggctgtnggtagatgtgcaatagaaatagctaatttatttccccaggtgtgtgctttaggcgtgggctgaccagtcttcttcctacatcttcttcccantaagtttcccctctggcttgacagcatgaggtgttgtgcattttttcagctcccccaggctgttctccaggcttcacagtctggtgcttgggagagtcaggcagggttaaactncaggagcagttt

SEQ ID NO: 22 - DNA, 298 bp, Homo sapiens
  misc_feature (92)..(92): May be any nucleotide
cttcatggaggaggtgcgccaggagctggaggacctggagaggagcctgactgaagagatggcgctgggggagcctgcggctgccgccgtgncactgctgggaggggaagagatttagatctggaccaggctgtgggtagatgtgcaatagaaatagctaatttatttccccaggtgtgtgctttaggcgtgggctgaccaggcttcttnctacatcttcttcccagtaagtttcccctctggcttgacagcatgaggtgttgtgcatttgttcagctcccccaggctgttctccagg

SEQ ID NO: 23 - DNA, 302 bp, Homo sapiens
  misc_feature (98)..(98): May be any nucleotide
gtcaagcccggcccggctctcagctacccgcagaggaggccaccctcaatgagatgttccgcgaggttgaggaactgatggaggacacgcagcacaanttgcgcangcggttggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagactggacaaatggtcttttcagagacagtt

SEQ ID NO: 24 - DNA, 279 bp, Homo sapiens
  misc_feature (52)..(52): May be any nucleotide
gaggagcctgactgaagagatggcgctgagggagcctgcggctgccgccgtngcactgctgggaggggaagagatttagatctggaccaggctgtgggtagatgtgcaatagaaatagctaatttatttccccaggtgtgtgctttaggcgtgggctgancaggcttcttnctacatcttcttgccagtangnttcccctctggcttgacagcatgaggtgttgtgcatttgttcagctcccccaggctgttctccaggcttcacagtctggtgcttgg

SEQ ID NO: 25 - DNA, 263 bp, Homo sapiens
  misc_feature (86)..(86): May be any nucleotide
taccatccatgtgcaccgagaaattcacaagataaccaacaaccagactggacaaatggtcttttcagagacagttatcacatctntgggagacgaagaaggcagaaggagccacgagtgcatcatcgacgaggactntgggcccagcatgtactgccagtttgccagcttccagtacacctgccagccatgccggggccagaggatgctctncacccgggacagtgagtgctgtggagaccagctgtgtgtctggggtcact

SEQ ID NO: 26 - DNA, 359 bp, Homo sapiens
  misc_feature (18)..(18): May be any nucleotide
ggctgcggcgcaagcgangatgcagcggcttggggccaccctgctgtgcctgctgctggcggcggcggtccccacggcccccgcgcccgctccgacggcgacctcggctccagtcaagcccggcccggctctcagctaccgcgcaggaggaggccaccctcaatgagatgttccgcgaggttgaggaactgatggaggacacgcagcacaaattggcaccggtggaagagatggaggcagaagaagctgctgctaaagcatcatcagaagtgaacctggcaaacttacctcccagctatcacaatgagaccaacacagacacgaaggttggaaataataccatccatgtgcaccgagaa

SEQ ID NO: 27 - DNA, 325 bp, Homo sapiens
  misc_feature (276)..(276): May be any nucleotide
accagagggactgccagccggggtgtgctcgtgccttccagagaggcctgctgttccctgtgtgcacacccctgcccgtggagcggacgctttgcatgaccccgccagccggcttctggacctcatcacctgggagctagagcctgatggagccttggaccgatgcccttgtgccagtggctcctctgccagccccacagccacagcctggtgtatgtgtgcaagccgaccttcgtggggagccgtgaccaagatggggagatcctgctgcccaanaaaggtccccgattgagtatgaagttggcaagcttcatggaaggaangg

SEQ ID NO: 28 - DNA, 238 bp, Homo sapiens
  misc_feature (121)..(121): May be any nucleotide
gagaaattcacaagataaccaacaaccagactggacaaatggtcttttcagagacagttatcacatctgtgggagacgaagaaggcagaaggagccacgagtgcatcatcgacgaggactntgggcccagcatgtactgccagtttnccagcttccagtacacctgccagccatgccggggccagaggatgctctgcacccgggacagtgagtgctgtggagaccagctgtgtgtctg

SEQ ID NO: 29 - DNA, 236 bp, Homo sapiens
  misc_feature (120)..(120): May be any nucleotide
ttggaaataataccatccatgtgcaccgagaaattcacaagataaccaacaaccagactggacaaatggtcttttcagagacagttatcacatctgtgggagacgaagaaggcagaaggngccacgagtgcatcatcgacgaggactgtgggcccagcatgtactgccagtttgccagcttccagtacacctgccagccatgtnggggccagaggatgctctgcacccgggacagt

SEQ ID NO: 30 - DNA, 344 bp, Homo sapiens
  misc_feature (155)..(155): May be any nucleotide
ccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggangctttgccatgaccccgccagccggcttctggacctcatcacctggggagctagagcctgatggagccttgggaccgatgcccttgtgccagtggcctcctcttgccagccccacagccacagcctgggtgtatgttgtgcaaagccgaccttcgtnggggaaccgtgaccaagatgggggagattctt

SEQ ID NO: 31 - DNA, 218 bp, Homo sapiens
  misc_feature (98)..(98): May be any nucleotide
ttttttggggaaataaattagctatttctattgcacatctacccacagcctggtccagatctaaatctcttcccctcccagcagtgcagcggcggcanaggnctcccccagcgccatctcttcagtcaggctcctctccaggtcctccagctcctggcgcacctcctccatgaagctgccaacttcatactcatcggggacctctctgggcagcagga

SEQ ID NO: 32 - DNA, 247 bp, Homo sapiens
gcagacggagatgcagcggcgttggggccaccctgactgtgcctgctgctggcggcggcggtccccacggcccccgcgcccgctccgacggcgacctcggctccagtcaagcccggcccggctctcagctacccgcaggagcgaggccaccctcaatgagatgttccgcgaggttgaggaactgatggaggacacgcagcacaaattgcgcagcggtgggaagagatggaggcagaagaagctgctg

SEQ ID NO: 33 - DNA, 210 bp, Homo sapiens
  misc_feature (5)..(5): May be any nucleotide
ctggnagaaggagcctgactgaaagagatggcgctgggggagcctgcggctgccgccgtgncactgctgggaggggaagagatttagatctggaccaggctgtgggtagatgtgcaatagaaatagctaatttatttccccaggtgtgtgctttaggcgtgggctgaccaggnttcttcctacatcttcttcccagtaagtttcccctct

SEQ ID NO: 34 - DNA, 303 bp, Homo sapiens
  misc_feature (51)..(51): May be any nucleotide
ccagtactggtacaatatggatcttttcagagacaggttatcacatctgtnggagacgaagaaggcagaaggagccacgagtgcatcatcgacgaggactgtgggcccggctctcagctacccgcagaggaggccaccctccthtagatgttccgcgagttgaggactgatggaggacacgctgcactgctgggaggggaagagatttagatctggaccaggctgtgggtagatgtgcaatagaaatagctaatttatttcccaggtgtgtgctttaggcgtggctgaccaggttcttctaca

SEQ ID NO: 35 - DNA, 174 bp, Homo sapiens
  misc_feature (130)..(130): May be any nucleotide
cgctgcactgctgggaggggaagagatttagatctggaccaggctgtgggtagatgtgcaatagaaatagctaatttatttccccaggtgtgtgctttaggcgtgggctgcccaggcttcttcctacatntccgtcccngtaagtttcccctctagcgaaaacagaataaggtg

SEQ ID NO: 36 - DNA, 151 bp, Homo sapiens
gacgaagaaggcagaaggagccacgagtgcatcatcgacgaggactgtgggccaagcatgtactgccagtttaacagctaacagtaccacctgccagccatgccggaaaaagaggatgactctgcacccgggacagtgagtgactgtagga

(19) United States
(12) Patent Application Publication
(10) Pub. No.: US 2003/0004327 A1
(43) Pub. Date: Jan. 2, 2003

(54) Cerebellum and embryo specific protein

(75) Inventors: Daniel R. Soppet, Centreville, VA (US); Steven M. Ruben, Olney, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Human Genome Sciences, Inc.

(21) Appl. No.: 10/201,310

(22) Filed: Jul. 24, 2002

Related U.S. Application Data
(63) Continuation of application No. 08/993,198, filed on Dec. 18, 1997.
(60) Provisional application No. 60/033,870, filed on Dec. 20, 1996.

(51) Int. Cl. C12P 21/02; C12N 5/06; C07H 21/04; C07K 14/475

(52) U.S. Cl. 536/23.2; 530/399; 435/69.1; 435/320.1; 435/325

(57) ABSTRACT

The present invention relates to a novel cerebellum and embryo specific (CESP) protein which is a member of the myocardial factor superfamily. In particular, isolated nucleic acid molecules are provided encoding the human CESP protein. CESP polypeptides are also provided as are vectors, host cells and recombinant methods for producing the same.

### CROSS-REFERENCE TO RELATED APPLICATIONS

This application claims priority benefit to U.S. Appl. No. 60/033,870, filed Dec. 20, 1996, which is hereby incorporated by reference.

### BACKGROUND OF THE INVENTION

1. Field of the Invention

The present invention relates to a novel endothelial factor. More specifically, isolated nucleic acid molecules are provided encoding a human cerebellum and embryo specific protein. Cerebellum and embryo specific polypeptides are also provided, as are vectors, host cells and recombinant methods for producing the same. Also provided are diagnostic and therapeutic methods relating to cerebellum and embryo specific protein-related disorders.

2. Related Art

Myocardial necrosis results from occlusion of a coronary artery by a thrombus, which forms on a destabilized atherosclerotic plaque, often following plaque rupture. Plaques most prone to rupture and thrombosis may initially be only mildly stenotic (i.e., 50% to 60% stenotic). However, myocardial damage proceeds rapidly as a “wave front” of injury, moving from endocardium to epicardium and may become complete and irreversible within three to four hours, unless the infarct zone is adequately nourished by collateral blood supply or unless recanalization of the artery (i.e., revascularization) is accomplished. See Rogers, W. J., Am. J. Med. 99:195-206 (1995). However, collateral circulation typically doesn't develop until a severe coronary artery stenosis has already developed (Schaper, W., European Heart J. 16:66-68 (1995)).

In one model of coronary angiogenesis, vascular formation occurs through three major stages including 1) vessel dilation and endothelial cell activation; 2) formation of a new vascular channel; and 3) maturation of the new vessel and final differentiation of all vascular cells (Rakusan, K., Coronary Angiogenesis: From Morphometry to Molecular Biology and Back, in: Claycomb, W. C and Di Nardo, P., eds., Ann New York Acad. Sci. 752:257-266 (1995)). Agents which promote angiogenesis, and particularly coronary artery angiogenesis, are therapeutically valuable to patients afflicted with vascular disease, and particularly heart disease. Such agents promote the formation of collateral circulation and ameliorate the pathological effects of coronary artery occlusion.

Percutaneous transluminal coronary angioplasty (PTCA) is commonly used revascularization treatment for coronary artery occlusion and myocardial necrosis. However, coronary artery luminal narrowing (restenosis) after PTCA is an unfortunate complication which occurs in many patients (Rensing, B. J. et al., Circulation 88:975-985 (1993)). There remains a need for therapeutic agents which can be used to prevent and treat restenosis.

### SUMMARY OF THE INVENTION

The present invention provides isolated nucleic acid molecules comprising a polynucleotide encoding the cerebellum and embryo specific protein (hereinafter “CESP”) having the amino acid sequence shown in SEQ ID NO:2 or the amino acid sequence encoded by the cDNA clone deposited in a bacterial host as ATCC Deposit Number 97728 on Sep. 23, 1996.

The present invention also relates to recombinant vectors, which include the isolated nucleic acid molecules of the present invention, and to host cells containing the recombinant vectors, as well as to methods of making such vectors and host cells and for using them for production of CESP polypeptides or peptides by recombinant techniques.

The invention further provides an isolated CESP polypeptide having an amino acid sequence encoded by a polynucleotide described herein.

For a number of CESP-related disorders, it is believed that significantly higher or lower levels of CESP gene expression can be detected in certain tissues (e.g., heart, renal tubule, renal glomerulus, vascular endothelium, and aortic endothelium) or bodily fluids (e.g., blood, serum, plasma, urine, synovial fluid or spinal fluid, and amniotic fluid) taken from an individual having such a disorder, relative to a “standard” CESP gene expression level, i.e., the CESP expression level in tissue or bodily fluids from an individual not having the CESP-related disorder. Thus, the invention provides a diagnostic method useful during diagnosis of a CESP-related disorder, which involves: (a) assaying CESP gene expression level in cells or body fluid of an individual; (b) comparing the CESP gene expression level with a standard CESP gene expression level, whereby an increase or decrease in the assayed CESP gene expression level compared to the standard expression level is indicative of a CESP-related disorder.

An additional aspect of the invention is related to a method for treating an individual in need of an increased level of CESP activity in the body comprising administering to such an individual a composition comprising a therapeutically effective amount of an isolated CESP polypeptide of the invention or an agonist thereof.

### BRIEF DESCRIPTION OF THE FIGURES

FIGS. 1A-1C show the nucleotide (SEQ ID NO:1) and deduced amino acid (SEQ ID NO:2) sequences of CESP. The protein has a leader sequence of about 21 amino acid residues (underlined) and a deduced molecular weight of about 38 kDa. The predicted amino acid sequence of the mature CESP protein is also shown.

FIG. 2 shows the regions of similarity between the amino acid sequences of the CESP protein and a chicken gene for which the function is unknown (Genbank accession number D26311; SEQ ID NO:3).

FIG. 3 shows an analysis of the CESP amino acid sequence. Alpha, beta, turn and coil regions; hydrophilicity and hydrophobicity; amphipathic regions; flexible regions; antigenic index and surface probability are shown. In the “Antigenic Index—Jameson-Wolf” graph, amino acid residues about 20 to about 86, about 92 to about 126, about 135 to about 157, about 169 to about 190, about 195 to about 219, about 234 to about 250, about 255 to about 274, and about 288 to about 336 in FIG. 1 correspond to the shown highly antigenic regions of the CESP receptor protein. These highly antigenic fragments in FIG. 1 correspond to the following fragments, respectively, in SEQ ID NO:2: amino acid residues about-I to about 65, about 71 to about 105, about 114 to about 136, about 148 to about 169, about 174 to about 198, about 213 to about 229, about 234 to about 253, and about 267 to about 315.

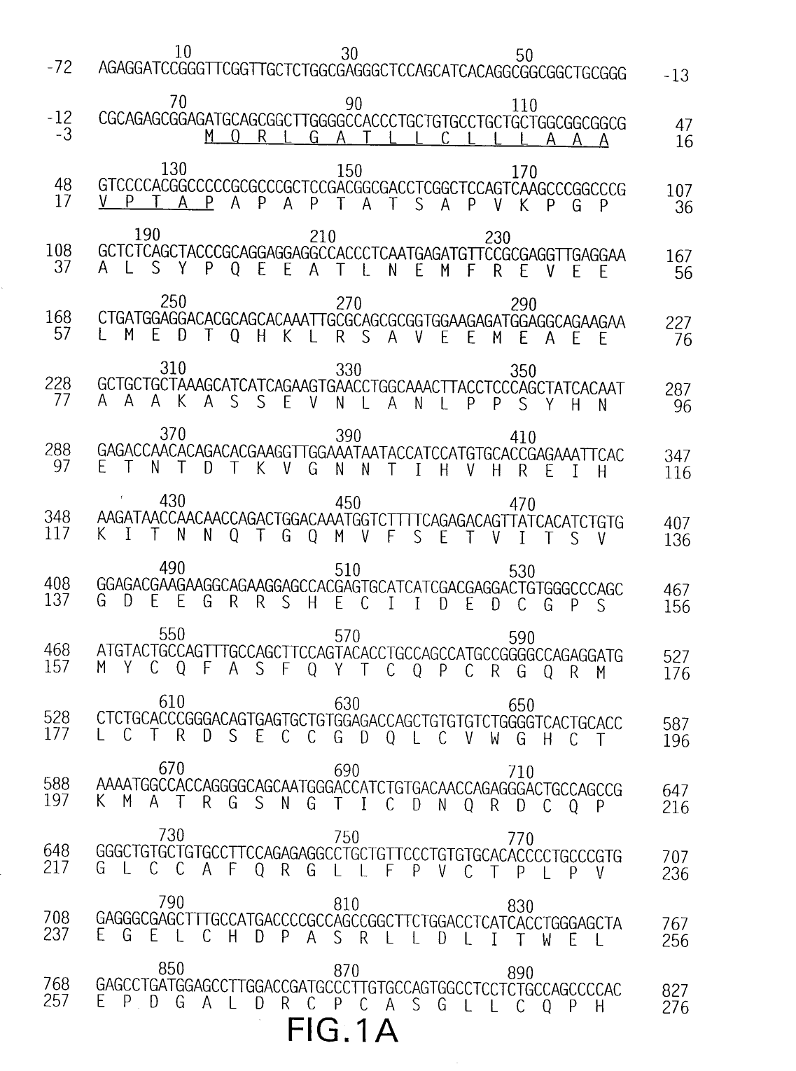

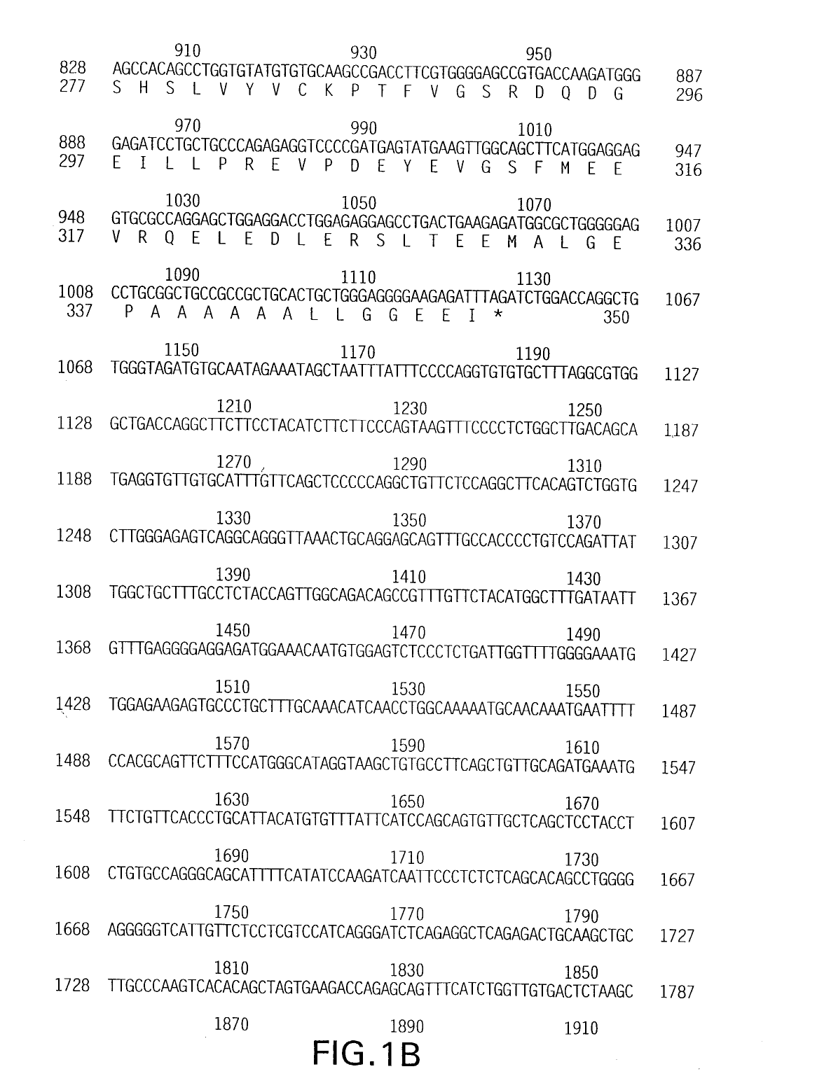

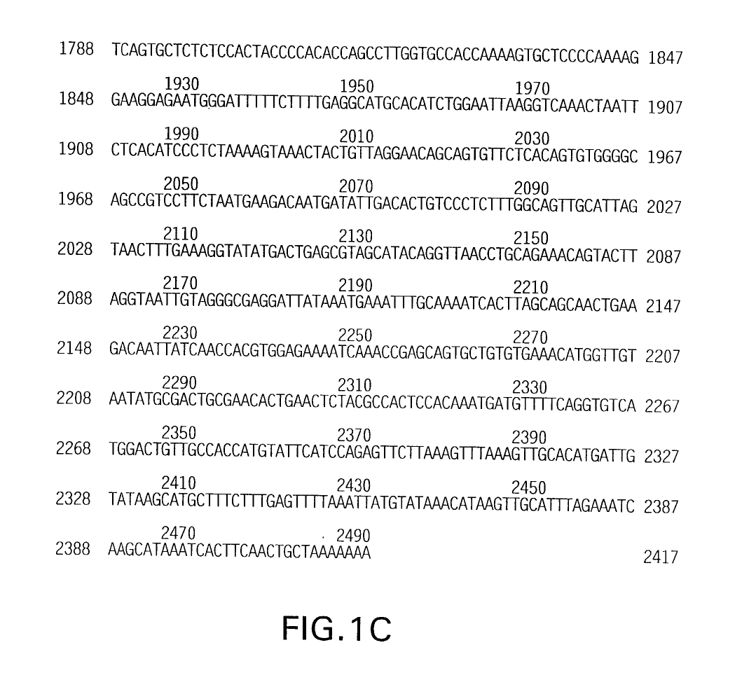

### DETAILED DESCRIPTION

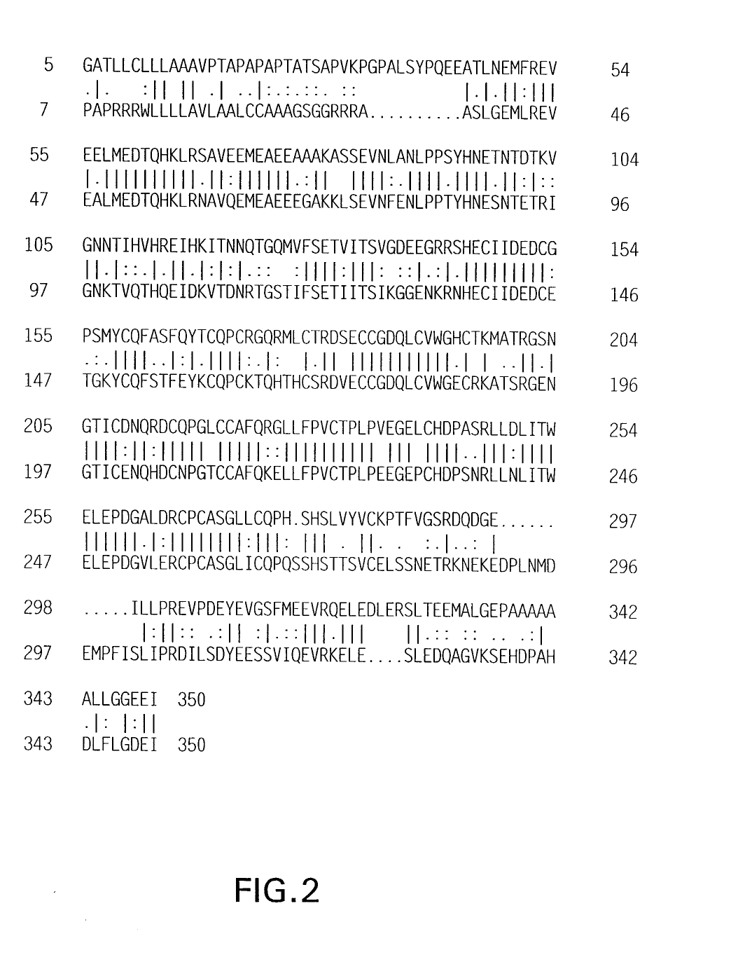

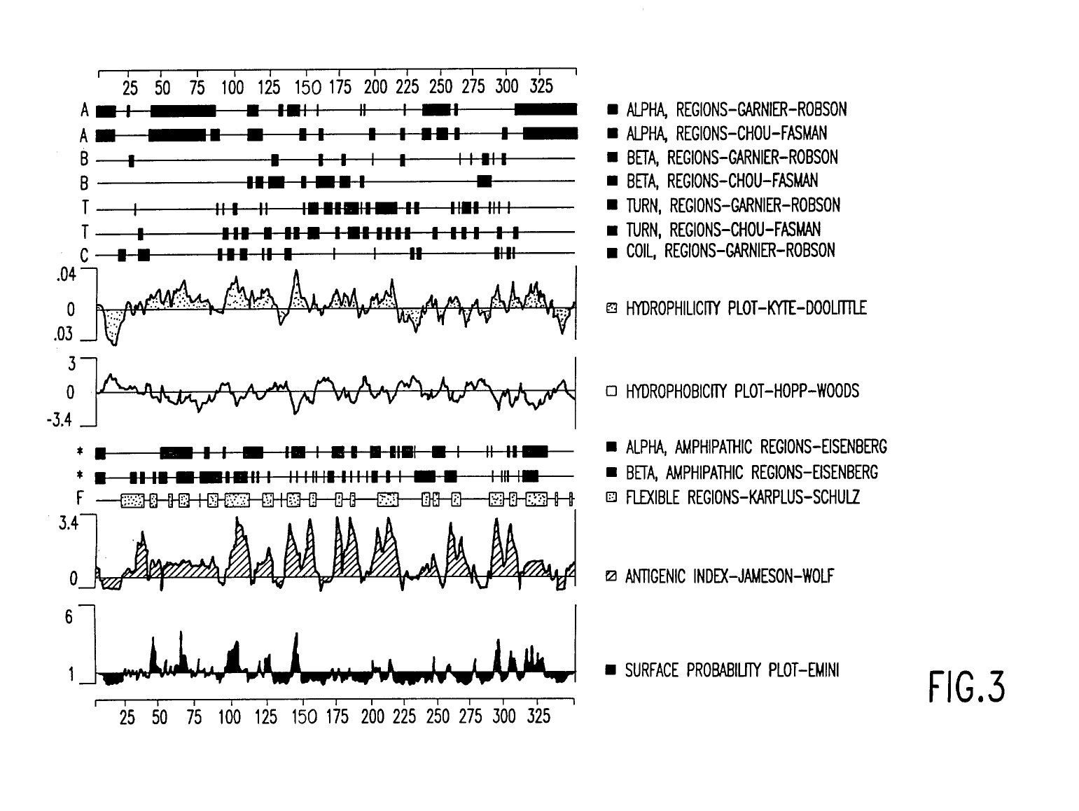

The present invention provides isolated nucleic acid molecules comprising a polynucleotide encoding a CESP polypeptide having the amino acid sequence shown in SEQ ID NO:2. The CESP protein of the present invention shares sequence homology with a chicken gene for which the function is unknown FIG. 2; SEQ ID NO:3) (Genbank accession number D26311).

The amino acid sequence in SEQ ID NO:2 was deduced from the sequence of CESP cDNA clone HHFHG78. The nucleotide sequence shown in SEQ ID NO:1 was obtained by sequencing the HHFHG78 clone, which was deposited on Sep. 23, 1996 at the American Type Culture Collection, 12301 Park Lawn Drive, Rockville, Md. 20852, and given accession number 97728. In this clone, the CESP sequence is contained between EcoR I and Xho I sites in the polylinker of the pBluescript SK(−) plasmid (Stratagene, La Jolla, Calif.).

Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence, compared to the actual sequence, will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in SEQ ID NO:1, a nucleic acid molecule of the present invention encoding a CESP polypeptide may be obtained using standard cloning and screening procedures, such as those for cloning cDNAs using mRNA as starting material. Illustrative of the invention, the nucleic acid molecule described in SEQ ID NO:1 was discovered in a cDNA library derived from human fetal heart. The determined nucleotide sequence of the CESP cDNA of SEQ ID NO:1 contains an open reading frame encoding a protein of 350 amino acid residues, and a deduced molecular weight of about 38 kDa. The CESP protein shown in SEQ ID NO:2 is about 58% identical and about 74% similar to an unknown chicken gene (Genbank accession number D26311) FIG. 2; SEQ ID NO:3).

The present invention also provides the mature form(s) of the CESP protein of the present invention. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature CESP polypeptides having the amino acid sequence encoded by the cDNA clone contained in the host identified as ATCC Deposit No. 97728 and as shown in SEQ ID NO:2. By the mature CESP protein having the amino acid sequence encoded by the cDNA clone contained in the host identified as ATCC Deposit 97728 is meant the mature form(s) of the CESP protein produced by expression in a mammalian cell (e.g., COS cells, as described below) of the complete open reading frame encoded by the human DNA sequence of the clone contained in the vector in the deposited host. As indicated below, the mature CESP protein having the amino acid sequence encoded by the cDNA clone contained in ATCC Deposit No. 97728 may or may not differ from the predicted “mature” CESP protein shown in SEQ ID NO:2 (amino acids from about 1 to about 329 in SEQ ID NO:2), depending on the accuracy of the predicted cleavage site based on computer analysis.

Methods for predicting whether a protein has a secretory leader as well as the cleavage point for that leader sequence are available. For instance, the methods of McGeoch (Virus Res. 3:271-286 (1985)) and von Heinje (Nucleic Acids Res. 14:4683-4690 (1986)) can be used. The accuracy of predicting the cleavage points of known mammalian secretory proteins for each of these methods is in the range of 75-80%. von Heinje, supra. However, the two methods do not always produce the same predicted cleavage point(s) for a given protein.

In the present case, the predicted amino acid sequence of the complete CESP polypeptides of the present invention were analyzed by a computer program (“PSORT”) (K. Nakai and M. Kanehisa, Genomics 14:897-911 (1992)), which is an expert system for predicting the cellular location of a protein based on the amino acid sequence. As part of this computational prediction of localization, the methods of McGeoch and von Heinje are incorporated. The analysis by the PSORT program predicted the cleavage sites between amino acids —1 and 1 in SEQ ID NO:2. Thereafter, the complete amino acid sequences were further analyzed by visual inspection, applying a simple form of the (−1,−3) rule of von Heinje. von Heinje, supra. Thus, the leader sequence for the CESP protein is predicted to consist of amino acid residues −21 to −1 in SEQ ID NO:2, while the predicted mature CESP protein consists of residues about 1 to about 329 in SEQ ID NO:2.

As one of ordinary skill would appreciate, due to the possibilities of sequencing errors discussed above, as well as the variability of cleavage sites for leaders in different known proteins, the full-length CESP polypeptide comprises about 350 amino acids, but may be anywhere in the range of 335 to 365 amino acids; and the predicted leader sequence of this protein is about 21 amino acids, but may be anywhere in the range of about 14 to about 50 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in SEQ ID NO:1; DNA molecules comprising the coding sequence for the mature CESP protein shown in SEQ ID NO:2 (last 329 amino acids); and DNA molecules which comprise a sequence substantially different from those described above but which, due to the degeneracy of the genetic code, still encode the CESP protein. Of course, the genetic code is well known in the art. Thus, it would be routine for one skilled in the art to generate such degenerate variants.

In another aspect, the invention provides isolated nucleic acid molecules encoding the CESP polypeptide having an amino acid sequence as encoded by the cDNA clone contained in the plasmid deposited as ATCC Deposit No. 97728 on Sep. 23, 1996. In further embodiments, this nucleic acid molecule will encode the mature polypeptide or the full-length polypeptide lacking the N-terminal methionine. The invention further provides an isolated nucleic acid molecule having the nucleotide sequence shown in SEQ ID NO:1 or the nucleic acid sequence of the CESP cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the CESP gene in human tissue, for instance, by Northern blot analysis.

The present invention is further directed to fragments of the isolated nucleic acid molecules described herein. By a fragment of an isolated nucleic acid molecule having the nucleotide sequence of the deposited cDNA or the nucleotide sequence shown in SEQ ID NO:1 is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50, 100, 150, 200, 250, 300, 350, 400, 350, 400, 450, 500, 550, 600, 650, 700, 750, 800, 850, 900, 950, 1000, 1050, or 1100 nt in length are also useful according to the present invention as are fragments corresponding to most, if not all, of the nucleotide sequence of the deposited cDNA or as shown in SEQ ID NO:1. By a fragment at least 20 nt in length, for example, is intended fragments which include 20 or more contiguous bases from the nucleotide sequence of the deposited cDNA or the nucleotide sequence as shown in SEQ ID NO:1.

Preferred nucleic acid fragments of the present invention also include nucleic acid molecules encoding epitope-bearing portions of the CESP protein. In particular, such nucleic acid fragments of the present invention include nucleic acid molecules encoding: a polypeptide comprising amino acid residues from about amino acid about −1 to about 65 in SEQ ID NO:2; a polypeptide comprising amino acid residues from about 71 to about 105 in SEQ ID NO:2; a polypeptide comprising amino acid residues from about 114 to about 136 in SEQ ID NO:2; a polypeptide comprising amino acid residues from about 148 to about 169 in SEQ ID NO:2; a polypeptide comprising amino acid residues from about 174 to about 198 in SEQ ID NO:2; a polypeptide comprising amino acid residues from about 213 to about 229 in SEQ ID NO:2; a polypeptide comprising amino acid residues from about 234 to about 253 in SEQ ID NO:2; and a polypeptide comprising amino acid residues from and about 267 to about 315 in SEQ ID NO:2.

In addition, the present inventors have identified the following cDNA clones related to extensive portions of the coding region of SEQ ID NO: 1: HHFBI55Ra (SEQ ID NO:12); HHFDB95R (SEQ ID NO:13); HUSFC71R (SEQ ID NO:14); and HCE2S01R (SEQ ID NO:15). The present inventors have identified the following cDNA clone related to an extensive portion of the non-coding region of SEQ ID NO:1: HCEB157R (SEQ ID NO:16) The following public ESTs, which relate to portions of the coding region of SEQ ID NO:1 have also been identified: GenBank Accession No. W61032 (SEQ ID NO:17); GenBank Accession No. AA349552 (SEQ ID NO:18); GenBank Accession No. R52311 (SEQ ID NO:19); GenBank Accession No. AA351624 (SEQ ID NO:20); GenBank Accession No. C₀₅₁₇₂ (SEQ ID NO:21); GenBank Accession No. T33818 (SEQ ID NO:22); GenBank Accession No. AA324686 (SEQ ID NO:23); GenBank Accession No. Z42237 (SEQ ID NO:24); GenBank Accession No. T30923 (SEQ ID NO:25); GenBank Accession No. AA226979 (SEQ ID NO:26); GenBank Accession No. W45085 (SEQ ID NO:27); GenBank Accession No. T31076 (SEQ ID NO:28); GenBank Accession No. T08793 (SEQ ID NO:29); GenBank Accession No. R14945 (SEQ ID NO:30); GenBank Accession No. AA031480 (SEQ ID NO:31); GenBank Accession No. AA424460 (SEQ ID NO:32); GenBank Accession No. C₀₅₂₉₆ (SEQ ID NO:33); GenBank Accession No. R58671 (SEQ ID NO:34); GenBank Accession No. T18925 (SEQ ID NO:35);and GenBank Accession No. R57834 (SEQ ID NO:36).

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit 97728. By “stringent hybridization conditions” is intended overnight incubation at 42° C. in a solution comprising: 50% formamide, 5× SSC (150 mM NaCl, 15 mM trisodium citrate), 50 mM sodium phosphate (pH 7.6), 5× Denhardt's solution, 10% dextran sulfate, and 20 g/ml denatured, sheared salmon sperm DNA, followed by washing the filters in 0. 1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30, 40, 50, 60, or 70 nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in SEQ ID NO:1). Of course, a polynucleotide which hybridizes only to a poly A sequence (such as the 3′ terminal poly(A) tract of the CESP cDNA shown in SEQ ID NO:1), or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a CESP polypeptide may include, but are not limited to those encoding the amino acid sequence of the mature polypeptide, by itself; the coding sequence for the mature polypeptide and additional sequences, such as those encoding the about 21 amino acid leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al, Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the CESP protein fused to Fc at the N- or C-terminus.

The present invention further relates to variants of the nucleic acid molecules of the present invention, which encode portions, analogs or derivatives of the CESP protein. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the CESP protein or portions thereof. Also especially preferred in this regard are conservative substitutions.

Further embodiments of the invention include isolated nucleic acid molecules comprising a polynucleotide having a nucleotide sequence at least 95% identical, and more preferably at least 96%, 97%, 98% or 99% identical to (a) a nucleotide sequence encoding the polypeptide having the complete amino acid sequence in SEQ ID NO:2 (amino acid residues −21 to 329), including the predicted leader sequence; (b) a nucleotide sequence encoding the polypeptide having the complete amino acid sequence in SEQ ID NO:2 except for the N-terminal methionine (amino acid residues −20 to 329); (c) a nucleotide sequence encoding the polypeptide having the amino acid sequence at positions 1-329 in SEQ ID NO:2; (d) a nucleotide sequence encoding the polypeptide having the complete amino acid sequence encoded by the cDNA clone contained in ATCC Deposit No. 97728; (e) a nucleotide sequence encoding the mature CESP polypeptide having the amino acid sequence encoded by the cDNA clone contained in ATCC Deposit No. 97728; or (f) a nucleotide sequence complementary to any of the nucleotide sequences in (a), (b), (c), (d), or (e).

By a polynucleotide having a nucleotide sequence at least, for example, 95% “identical” to a reference nucleotide sequence encoding a CESP polypeptide is intended that the nucleotide sequence of the polynucleotide is identical to the reference sequence except that the polynucleotide sequence may include up to five point mutations per each 100 nucleotides of the reference nucleotide sequence encoding the CESP polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 95%, 96%, 97%, 98% or 99% identical to, for instance, the nucleotide sequence shown in SEQ ID NO:1 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 95%, 96%, 97%, 98% or 99% identical to the nucleic acid sequence shown in SEQ ID NO: 1 or to the nucleic acid sequence of the deposited cDNA. This is because even where a particular nucleic acid molecule does not encode a polypeptide having CESP activity, one of skill in the art would still know how to use the nucleic acid molecule, for instance, as a hybridization probe or a polymerase chain reaction (PCR) primer. Uses of the nucleic acid molecules of the present invention that do not encode a polypeptide having CESP activity include, inter alia, (1) isolating the CESP gene or allelic variants thereof in a cDNA library; (2) in situ hybridization (e.g., “FISH”) to metaphase chromosomal spreads to provide precise chromosomal location of the CESP gene, as described in Verma et al., Human Chromosomes: A Manual of Basic Techniques, Pergamon Press, New York (1988); and (3) Northern Blot analysis for detecting CESP mRNA expression in specific tissues.

Preferred, however, are nucleic acid molecules having sequences at least 95%, 96%, 97%, 98% or 99% identical to the nucleic acid sequence shown in SEQ ID NO: 1 or to the nucleic acid sequence of the deposited cDNA which do, in fact, encode a polypeptide having CESP activity. By “a polypeptide having CESP activity” is intended polypeptides exhibiting activity in a particular biological assay. For example, protein activity can be measured using the morphometrically quantitative in vitro assay for angiogenesis as described by Sueishi et al. (Japanese Circulation J. 56:192-198 (1992). This assay utilizes a model of angiogenesis in a culture system using type I collagen gel s a reconstructed subendothelial matrix. The length of capillary-like tubular structures are measured morphometrically using an image analyzer. Briefly, this assay involves isolating and culturing capillary endothelial cells (for example, from bovine adrenal cortex or another suitable source), administering a candidate protein to the cell culture, and measuring morphometrically the total length of tubular structures using phase-contrast microscopic photography.

Of course, due to the degeneracy of the genetic code, one of ordinary skill in the art will immediately recognize that a large number of the nucleic acid molecules having a sequence at least 95%, 96%, 97%, 98% or 99% identical to the nucleic acid sequence shown in SEQ ID NO:1 or to the nucleic acid sequence of the deposited cDNA will encode a polypeptide having CESP protein activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having CESP protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Vectors and Host Cells

The present invention also relates to vectors which include the isolated DNA molecules of the present invention, host cells which are genetically engineered with the recombinant vectors, and the production of CESP polypeptides or fragments thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate heterologous hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2,045,869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5. See, D. Bennett et al., Journal of Molecular Recognition, Vol. 8:52-58 (1995) and K. Johanson et al., The Journal of Biological Chemistry, Vol. 270, No. 16:9459-9471 (1995).

The CESP protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

CESP Polypeptides and Fragments

The invention further provides an isolated CESP polypeptide having the amino acid sequence encoded by the deposited cDNA, or the amino acid sequence in SEQ ID NO:2, or a peptide or polypeptide comprising a portion of the above polypeptides.

It will be recognized in the art that some amino acid sequences of the CESP polypeptide can be varied without significant effect of the structure or function of the protein. If such differences in sequence are contemplated, it should be remembered that there will be critical areas on the protein which determine activity.

Thus, the invention further includes variations of the CESP polypeptide which show substantial CESP polypeptide activity or which include regions of CESP protein such as the protein portions discussed below. Such mutants include deletions, insertions, inversions, repeats, and type substitutions. As indicated above, guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the CESP protein. The prevention of aggregation is highly desirable. Aggregation of proteins not only results in a loss of activity but can also be problematic when preparing pharmaceutical formulations, because they can be immunogenic (Pinckard et al, Clin. Exp. Immunol. 2:331-340 (1967); Robbins et al., Diabetes 36:838-845 (1987); and Cleland et al. Crit. Rev. Therapeutic Drug Carrier Systems 10:307-377 (1993)).

The replacement of amino acids can also change the selectivity of binding to cell surface receptors. Ostade et al., Nature 361:266-268 (1993), describes certain mutations resulting in selective binding of TNF-α to only one of the two known types of TNF receptors. Thus, the CESP protein of the present invention may include one or more amino acid substitutions, deletions or additions, either from natural mutations or human manipulation.

As indicated, changes are preferably of a minor nature, such as conservative amino acid substitutions that do not significantly affect the folding or activity of the protein (see Table 1).

Amino acids in the CESP protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Of course, the number of amino acid substitutions a skilled artisan would make depends on many factors, including those described above. Generally speaking, the number of amino acid substitutions for any given CESP polypeptide will not be more than 50, 40, 30, 20, 10, 5, or 3.

Amino acids in the CESP protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity, such as in vitro proliferative activity.

The polypeptides of the present invention are preferably provided in an isolated form. By “isolated polypeptide” is intended a polypeptide removed from its native environment. Thus, a polypeptide produced or contained in a recombinant host cell is considered “isolated” for the purposes of the present invention. Also intended as “isolated” is a polypeptide that has been purified, partially or substantially, from a recombinant host or from a native source. For example, a recombinantly produced version of the CESP polypeptide can be substantially purified by the one-step method described in Smith and Johnson, Gene 67:31-40 (1988).

The polypeptides of the present invention include the complete polypeptide encoded by the deposited cDNA; the mature polypeptide encoded by the deposited cDNA; amino acid residues −21 to 329 of SEQ ID NO:2; amino acid residues −20 to 329 of SEQ ID NO:2; and amino acid residues 1 to 329 in SEQ ID NO:2, as well as polypeptides which are at least 95% identical, and more preferably at least 96%, 97%, 98% or 99% identical to the polypeptide encoded by the deposited cDNA, and to the polypeptides of SEQ ID NO:2, and also include portions of such polypeptides with at least 30 amino acids and more preferably at least 50 amino acids.

By a polypeptide having an amino acid sequence at least, for example, 95% “identical” to a reference amino acid sequence of a CESP polypeptide is intended that the amino acid sequence of the polypeptide is identical to the reference sequence except that the polypeptide sequence may include up to five amino acid alterations per each 100 amino acids of the reference amino acid of the CESP polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 95%, 96%, 97%, 98% or 990% identical to, for instance, the amino acid sequence shown in SEQ ID NO:2 or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

In another aspect, the invention provides a peptide or polypeptide comprising an epitope-bearing portion of a polypeptide of the invention. The epitope of this polypeptide portion is an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in the art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A., Antibodies that react with predetermined sites on proteins, Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777.

Antigenic epitope-bearing peptides and polypeptides of the invention preferably contain a sequence of at least seven, more preferably at least nine and most preferably between about at least about 15 to about 30 amino acids contained within the amino acid sequence of a polypeptide of the invention.

Non-limiting examples of antigenic polypeptides or peptides that can be used to generate CESP-specific antibodies include: a polypeptide comprising amino acid residues from about amino acid about −1 to about 65 in SEQ ID NO:2; a polypeptide comprising amino acid residues from about 71 to about 105 in SEQ ID NO:2; a polypeptide comprising amino acid residues from about 114 to about 136 in SEQ ID NO:2; a polypeptide comprising amino acid residues from about 148 to about 169 in SEQ ID NO:2; a polypeptide comprising amino acid residues from about 174 to about 198 in SEQ ID NO:2; a polypeptide comprising amino acid residues from about 213 to about 229 in SEQ ID NO:2; a polypeptide comprising amino acid residues from about 234 to about 253 in SEQ ID NO:2; and a polypeptide comprising amino acid residues from and about 267 to about 315 in SEQ ID NO:2.

The epitope-bearing peptides and polypeptides of the invention may be produced by any conventional means (Houghten, R. A., General method for the rapid solid-phase synthesis of large numbers of peptides: Specificity of antigen-antibody interaction at the level of individual amino acids, Proc. Natl. Acad. Sci. USA 82:5131-5135 (1985)). This “Simultaneous Multiple Peptide Synthesis (SMPS)” process is further described in U.S. Pat. No. 4,631,211 to Houghten et al. (1986).

As one of skill in the art will appreciate, CESP polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric CESP protein or protein fragment alone (Fountoulakis et al., J Biochem. 270:3958-3964 (1995)).

Diagnostic and Prognostic Applications of CESP

It is believed that certain tissues in mammals with a CESP-related disorder express significantly enhanced or diminished levels of the CESP protein and mRNA encoding the CESP protein when compared to a corresponding “standard” mammal, i.e., a mammal of the same species not having the disorder. Further, it is believed that enhanced or diminished levels of the CESP protein can be detected in certain body fluids (e.g., blood, sera, plasma, urine, and spinal fluid) from mammals with the disorder when compared to sera from mammals of the same species not having the disorder. Thus, the invention provides a diagnostic method useful during diagnosis, which involves assaying the expression level of the gene encoding the CESP protein in mammalian cells or body fluid and comparing the gene expression level with a standard CESP gene expression level, whereby an increase in the gene expression level over the standard is indicative of certain disorders.

CESP related disorders include but are not limited to coronary restenosis following coronary revascularization, coronary artery thrombus or occlusion, myocardial infarction, atrial and/or ventricular arrhythmias, heart block, hereditary medial “necrosis” of small coronary and pulmonary arteries, focal fibromuscular dysplasia of small coronary arteries, cardiomyopathy, arrhythmogenic right ventricular dysplasia, and sudden death.

Where a diagnosis has already been made according to conventional methods, the present invention is useful as a prognostic indicator, whereby patients exhibiting enhanced or decreased CESP gene expression will experience a worse clinical outcome relative to patients expressing the gene at a lower level. Further, CESP is detected in amniotic cells. It is believed that CESP can serve as a marker for fetal genetic defects. Such fetal genetic defects include developmental cardiac defects.

By “assaying the expression level of the gene encoding the CESP protein” is intended qualitatively or quantitatively measuring or estimating the level of the CESP protein or the level of the mRNA encoding the CESP protein in a first biological sample either directly (e.g., by determining or estimating absolute protein level or mRNA level) or relatively (e.g., by comparing to the CESP protein level or mRNA level in a second biological sample).

Preferably, the CESP protein level or mRNA level in the first biological sample is measured or estimated and compared to a standard CESP protein level or mRNA level, the standard being taken from a second biological sample obtained from an individual not having the disorder. As will be appreciated in the art, once a standard CESP protein level or mRNA level is known, it can be used repeatedly as a standard for comparison.

By “biological sample” is intended any biological sample obtained from an individual, cell line, tissue culture, or other source which contains CESP protein or mRNA. Biological samples include mammalian body fluids (such as blood, sera, plasma, urine, synovial fluid, spinal fluid, and amniotic fluid containing amniotic cells) which contain secreted mature CESP protein, heart, renal glomerulus, and renal tubule.

The present invention is useful for detecting CESP-related disorders in mammals. Preferred mammals include monkeys, apes, cats, dogs, cows, pigs, horses, rabbits and humans. Particularly preferred are humans.

Total cellular RNA can be isolated from a biological sample using the single-step guanidinium-thiocyanate-phenol-chloroform method described in Chomczynski and Sacchi, Anal Biochem. 162:156-159 (1987). Levels of mRNA encoding the CESP protein are then assayed using any appropriate method. These include Northern blot analysis Harada et al., Cell 63:303-312 (1990)), S1 nuclease mapping (Fujita et al., Cell 49:357-367 (1987)), the polymerase chain reaction (PCR), reverse transcription in combination with the polymerase chain reaction (RT-PCR) (Makino et al., Technique 2:295-301 (1990)), and reverse transcription in combination with the ligase chain reaction (RT-LCR).

Assaying CESP protein levels in a biological sample can occur using antibody-based techniques. For example, CESP protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)).

Other antibody-based methods useful for detecting CESP protein gene expression include immunoassays, such as the enzyme linked immunosorbent assay (ELISA) and the radioimmunoassay (RIA). Suitable labels are known in the art and include enzyme labels, such as glucose oxidase, and radioisotopes, such as iodine (¹²⁵I, ¹²¹I), carbon (¹⁴C), sulphur (³⁵S), tritium (³H), indium (¹¹²In), and technetium (^{99m}Tc), and fluorescent labels, such as fluorescein and rhodamine, and biotin.

CESP Protein Therapy

It is believed that HSF plays a role in a wide variety of physiologic and pathologic processes. Accordingly, the CESP protein has application to any physiologic or pathologic disease condition in which abnormal activity of the CESP system is implicated and has pathological or physiological consequences.

Physiological processes in which CESP is believed to be involved include the regulation of collateral circulation (particularly in the heart) regulation of coronary artery restenosis following a revascularization procedure, regulation of apoptosis in myocytes, the modulation of myocyte development in the developing heart, regulation of circulating blood volume, regulation of vascular tone, regulation of blood pressure and cardiac output, diuresis, natriuresis, facilitation of transudation of plasma water to the interstitium, and inhibition of the release or action of hormones such as aldosterone, angiotensin II, endothelins, renin, and vasopressin.

It is also believed that CESP plays a role as a growth modulator in the developing heart. Moreover, it is believed that CESP protects adult myocardial cells from damage during myocardial ischemia. Further, it is believed that CESP enhances revascularization of cardiac muscle following revascularization therapy (e.g., coronary artery bypass surgery; percutaneous transluminal coronary angioplasty; or administration of an anticoagulant such as heparin, hirudin, urokinase, streptokinase, or tissue plasminogen activator) and prevents or inhibits restenosis of coronary arteries following revascularization therapy. Accordingly, when CESP is administered to a patient receiving revascularization therapy, CESP enhances revascularization of cardiac muscle and prevents or inhibits restenosis of coronary arteries.

It is also believed that CESP facilitates angiogenesis (i.e., the formation of new vascular tissue). Accordingly, administration of CESP to patients afflicted by circulatory illnesses facilitates angiogenesis. Circulatory illness for which CESP treatment is beneficial include atherosclerotic heart disease, coronary artery constriction, coronary artery blockage (either partial or full), myocardial infarction, venous thrombosis, and Reynaud's syndrome.

The present invention is useful for treating or preventing CESP-related disorders in mammals. Preferred mammals include monkeys, apes, cats, dogs, cows, pigs, horses, rabbits and humans. Particularly preferred are humans.

Modes of Administration

It will be appreciated that conditions caused by a decrease in the standard or normal level of CESP activity in an individual, can be treated by administration of CESP protein. Thus, the invention further provides a method of treating an individual in need of an increased level of CESP activity comprising administering to such an individual a pharmaceutical composition comprising an effective amount of an isolated CESP polypeptide of the invention, particularly a mature form of the CESP, effective to increase the CESP activity level in such an individual.

As a general proposition, the total pharmaceutically effective amount of CESP polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the CESP polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed.

Pharmaceutical compositions containing the CESP protein of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Chromosome Assays

The nucleic acid molecules of the present invention are also valuable for chromosome identification. The sequence is specifically targeted to and can hybridize with a particular location on an individual human chromosome. The mapping of DNAs to chromosomes according to the present invention is an important first step in correlating those sequences with genes associated with disease.

In certain preferred embodiments in this regard, the cDNA herein disclosed is used to clone genomic DNA of a CESP protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes.

Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

### Example 1

### Expression and Purification of CESP in E. coli

The bacterial expression vector pQE60 is used for bacterial expression in this example. (QIAGEN, Inc., 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Mi-NTA”) affinity resin sold by QIAGEN, Inc., supra, and suitable single restriction enzyme cleavage sites. These elements are arranged such that an inserted DNA fragment encoding a polypeptide expresses that polypeptide with the six His residues (i.e., a “6× His tag”) covalently linked to the carboxyl terminus of that polypeptide.

The DNA sequence encoding the desired portion of the CESP protein lacking the hydrophobic leader sequence is amplified from the deposited cDNA clone using PCR oligonucleotide primers which anneal to the amino terminal sequences of the desired portion of the CESP protein and to sequences in the deposited construct 3′ to the cDNA coding sequence. Additional nucleotides containing restriction sites to facilitate cloning in the pQE60 vector are added to the 5′ and 3′ sequences, respectively.

For cloning the mature protein, the 5′ primer has the sequence 5′-GGGA-GGATCCGCGCCCGCTCCGACGGCG-3′ (SEQ ID NO:4), containing the underlined BamH I restriction site followed by 20 nucleotides corresponding to nucleotides 134-153 of the CESP cDNA sequence set out in SEQ ID NO:1. One of ordinary skill in the art would appreciate, of course, that the point in the protein coding sequence where the 5′ primer begins may be varied to amplify a DNA segment encoding any desired portion of the complete protein shorter or longer than the mature form. The 3′ primer has the sequence 5′-GCCTCTAGATTAAATCTCTTCCCCTCCCAGCAGT-3′ (SEQ ID NO:5), containing the underlined Xba I restriction site followed by 24 nucleotides complementary to nucleotides 1101 to 1124 of the CESP DNA sequence set out in SEQ ID NO:1, with the coding sequence aligned with the restriction site so as to maintain its reading frame with that of the six His codons in the pQE60 vector.

The amplified CESP DNA fragment and the vector pQE60 are digested with BamH I and Xba I restriction enzymes and the digested DNAs are then ligated together. Insertion of the CESP DNA into the restricted pQE60 vector places the CESP protein coding region downstream from the IPTG-inducible promoter and in-frame with an initiating AUG and the six histidine codons.

The ligation mixture is transformed into competent E. coli cells using standard procedures such as those described in Sambrook et al., Molecular Cloning: a Laboratory Manual, 2nd Ed; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kan^{r}”), is used in carrying out the illustrative example described herein. This strain, which is only one of many that are suitable for expressing CESP protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 34 hours at 4° C. in 6M guanidine-HCl, pH 8. The cell debris is removed by centrifugation, and the supernatant containing the CESP protein is loaded onto a nickel-nitrilo-tri-acetic acid (“NiNTA”) affinity resin column (available from QIAGEN, Inc., supra). Proteins with a 6× His tag bind to the NI-NTA resin with high affinity and can be purified in a simple one-step procedure (for details see: The QIAexpressionist, 1995, QIAGEN, Inc., supra). Briefly the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the CESP protein is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins can be eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

### Example 2

### Cloning and Expression of CESP Protein in a Baculovirus Expression System

The cDNA sequence encoding the full length CESP protein in the deposited clone was amplified using PCR oligonucleotide primers corresponding to the 5′ and 3′ sequences of the gene:

The 5′ primer had the sequence 5′-TGCCGCGGATCCGCCATCATG CAGCGGCTTGGGGCCAC-3′ (SEQ ID NO:6), containing the underlined BamH I restriction enzyme site followed by 20 nucleotides corresponding to nucleotides 73-92 of the CESP protein coding sequence set out in SEQ ID NO:1. Inserted into an expression vector, as described below, the 5′ end of the amplified fragment encoding CESP provided an efficient signal peptide. An efficient signal for initiation of translation in eukaryotic cells, as described by Kozak, M., J. Mol. Biol. 196:947-950 (1987) was appropriately located in the vector portion of the construct.

The 3′ primer had the sequence 5′GCACAGGTACCCACAGCCTGGTC-CAGATCTAAATCTCTTCCCCTCCCAG 3′ (SEQ ID NO:7), containing the underlined Asp718 restriction site followed by 42 nucleotides complementary to nucleotides 1105-1145 of the CESP cDNA sequence set out in SEQ ID NO:1.

The amplified fragment was isolated from a 1% agarose gel using a commercially available kit (“Geneclean,” BIO 101 Inc., La Jolla, Calif.). The fragment then was digested with BamH I and Asp718 and again was purified on a 1% agarose gel. This fragment is designated herein F2.

The vector pA2 was used to express the CESP protein in the baculovirus expression system, using standard methods, as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites. The polyadenylation site of the simian virus 40 (“SV40”) is used for efficient polyadenylation. For an easy selection of recombinant virus the beta-galactosidase gene from E. coli is inserted in the same orientation as the polyhedrin promoter and is followed by the polyadenylation signal of the polyhedrin gene. The polyhedrin sequences are flanked at both sides by viral sequences for cell-mediated homologous recombination with wild-type viral DNA to generate a viable virus that expresses the cloned polynucleotide.

The pA2 expression vector contains the strong polyhedrin promoter of the Autographa californica nuclear polyhedrosis virus (AcMNPV) followed by convenient restriction sites. The polyadenylation site of the simian virus 40 (“SV40”) is used for efficient polyadenylation. For an easy selection of recombinant virus the beta-galactosidase gene from E. coli is inserted in the same orientation as the polyhedrin promoter and is followed by the polyadenylation signal of the polyhedrin gene. The polyhedrin sequences are flanked at both sides by viral sequences for cell-mediated homologous recombination with wild-type viral DNA to generate viable virus that express the cloned polynucleotide.

Many other baculovirus vectors could be used in place of pA2, such as pA2-GP (which contains the AcMNPV gp 67 signal peptide), pAc373, pVL941 and pAcIM1 provided, as those of skill readily will appreciate, that construction provides appropriately located signals for transcription, translation, trafficking and the like, such as an in-frame AUG and a signal peptide, as required. Such vectors are described in Luckow et al., Virology 170: 31-39, among others.

The pA2 plasmid was digested with the restriction enzymes BamH I and Asp718. The DNA was then isolated from a 1% agarose gel using a commercially available kit (“Geneclean” BIO 101 Inc., La Jolla, Calif.). This vector DNA is designated herein “V”.

Fragment F2 and the dephosphorylated plasmid V2 were ligated together with T4 DNA ligase. E. coli HB 101 cells were transformed with ligation mix and spread on culture plates. Bacteria were identified that contained the plasmid with the human CESP gene by digesting DNA from individual colonies using BamH I and Asp718 and then analyzing the digestion product by gel electrophoresis. The sequence of the cloned fragment was confirmed by DNA sequencing. This plasmid is designated herein pBacCESP.

5 μg of the plasmid pBacCESP were co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). 1 μg of BaculoGold™ virus DNA and 5 μg of the plasmid pBacCESP were mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture was added drop-wise to Sf9 insect cells (ATCC CRL 1711) seeded in a 35 mm tissue culture plate with 1 μl Grace's medium without serum. The plate was rocked back and forth to mix the newly added solution. The plate was then incubated for 5 hours at 27° C. After 5 hours, the transfection solution was removed from the plate and 1 ml of Grace's insect medium supplemented with 10% fetal calf serum was added. The plate was put back into an incubator and cultivation was continued at 27° C. for four days.

After four days, the supernatant was collected and a plaque assay was performed, as described by Summers and Smith, cited above. An agarose gel with “Blue Gal” (Life Technologies Inc., Gaithersburg) was used to allow easy identification and isolation of gal-expressing clones, which produced blue-stained plaques. (A detailed description of a “plaque assay” of this type can also be found in the user's guide for insect cell culture and baculovirology distributed by Life Technologies Inc., Gaithersburg, page 9-10).

Four days after serial dilution, the virus was added to the cells. After appropriate incubation, blue stained plaques were picked with the tip of an Eppendorf pipette. The agar containing the recombinant viruses was then resuspended in an Eppendorf tube containing 200 μl of Grace's medium. The agar was removed by a brief centrifugation and the supernatant containing the recombinant baculovirus was used to infect Sf9 cells seeded in 35 mm dishes. Four days later, the supernatants of these culture dishes were harvested and then they were stored at 4° C. A clone containing properly inserted hESSB I, II and III was identified by DNA analysis including restriction mapping and sequencing. This is designated herein as V-CESP.

Sf9 cells were grown in Grace's medium supplemented with 10% heat-inactivated FBS. The cells were infected with the recombinant baculovirus V-CESP at a multiplicity of infection (“MOI”) of about 2 (about 1 to about 3). Six hours later, the medium was removed and was replaced with SF900 II medium minus methionine and cysteine (available from Life Technologies Inc., Gaithersburg). 42 hours later, 5 μCi of ³⁵S-methionine and 5 μCi ³⁵S-cysteine (available from Amersham) were added. The cells were further incubated for 16 hours and then they were harvested by centrifugation, lysed and the labeled proteins were visualized by SDS-PAGE and autoradiography.

### Example 3

### Cloning and Expression in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from retroviruses, e.g., RSV, HTLVI, HVI and the early promoter of the cytomegalovirus (CMV). However, cellular elements can also be used (e.g., the human actin promoter). Suitable expression vectors for use in practicing the present invention include, for example, vectors such as PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human HeLa 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites BamH I, Xba I and Asp 718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pCESP HA, is made by cloning a cDNA encoding CESP into the expression vector pcDNAI/Amp or pcDNA3 (which can be obtained from Invitrogen, Inc.).

The expression vector pcDNA3 contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37:767 (1984). The fusion of the HA tag to the target protein allows easy detection and recovery of the recombinant protein with an antibody that recognizes the HA epitope. pcDNA3 contains, in addition, the selectable neomycin marker.

A DNA fragment encoding the CESP is cloned into the polylinker region of the vector so that recombinant protein expression is directed by the CMV promoter. The plasmid construction strategy is as follows. The CESP cDNA of the deposited clone is amplified using primers that contain convenient restriction sites, much as described above for construction of vectors for expression of CESP in E. coli. Suitable primers include the following, which are used in this example. The 5′ primer has the sequence 5′-TGCCGCGGATCCGCCATCATG CAGCGGCTTGGGGCCAC-3′ (SEQ ID NO:6), containing the underlined BamH I restriction enzyme site, a Kozak sequence, and an AUG start codon followed by 20 nucleotides (nucleotides 73-92) of the CESP protein coding sequence in SEQ ID NO:1. If no HA tag is used, the 3′ primer has the sequence 5′-GTCTCTAGACAGATCTAAATCTCTTCCCCTCCCAG-3′ (SEQ ID NO:8), containing the underlined Xba I site and 26 nucleotides complementary to nucleotides 1105-1130 of the CESP cDNA sequence set out in SEQ ID NO: 1. If an HA tag is used, the 3′ primer has the sequence 5′-GTCTCTAGACAGA-TCTAAGCGTAGTCTGGGACGTCGTATGGGTAAATCTCTTCCCCTCCC-AGCAG-3′ (SEQ ID NO:9), containing the underlined Xba I site and 23 nucleotides complementary to nucleotides 1102-1124 of the CESP cDNA sequence set out in SEQ ID NO:1

The PCR amplified DNA fragment and the vector, pcDNA3, are digested with Xba I restriction enzyme and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the CESP-encoding fragment.

For expression of recombinant CESP, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of CESP by the vector.

Expression of the CESP-HA fusion protein is detected by radiolabeling and immunoprecipitation, using methods described in, for example Harlow et al., Antibodies: A Laboratory Manual, 2nd Ed; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and lysed with detergent-containing RIPA buffer: 150 mM NaCl, 1% NP-40, 0.1% SDS, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 was used for the expression of the CESP protein. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are BamH I, Xba I, and Asp 718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the CESP protein in a regulated way in mammalian cells (Gossen, M., & Bujard, H. 1992, Proc. Natl. Acad. Sci. USA 89: 5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 was digested with the restriction enzyme BamH I and then dephosphorylated using calf intestinal phosphatase by procedures known in the art. The vector was then isolated from a 1% agarose gel.

The DNA sequence encoding the complete CESP protein, including its leader sequence, was amplified using PCR oligonucleotide primers corresponding to the 5′ and 3′ sequences of the gene. The 5′ primer had the sequence 5′-GC TGCCGCGGATCCGCCACCATGCAGCGGCTTGGGGCCACC 3′ (SEQ ID NO:10 containing the underlined BamH I restriction enzyme site, an efficient signal for initiation of translation in eukaryotes, as described by Kozak, M., J. Mol. Biol. 196:947-950 (1987), and followed by 21 nucleotides corresponding to nucleotides 73-93 of the CESP protein coding sequence set out in SEQ ID NO:1.

The 3′ primer had the sequence 5′-CACACGCGGATCCAGATCTAAA TCTCTTCCCCTC-3′ (SEQ ID NO:11) containing the underlined BamH I restriction site followed by 24 nucleotides (nucleotides 1109-1132) complementary to the CESP protein coding sequence set out in SEQ ID NO:1, including the stop codon.

The amplified fragment was digested with the restriction enzyme BamH I and then purified again on a 1% agarose gel. The isolated fragment and the dephosphorylated vector were then ligated with T4 DNA ligase. E. coli HB101 or XL-1 Blue cells were then transformed and bacteria were identified that contain the fragment inserted into plasmid pC4 using restriction enzyme analysis.

Chinese hamster ovary cells lacking an active DHFR gene were used for transfection. 5 μg of the expression plasmid pC4 were cotransfected with 0.5 μg of the plasmid pSV2-neo using lipofectin (Felgner et al., supra). The plasmid pSV2-neo contains a dominant selectable marker, the neo gene from Tn5 encoding an enzyme that confers resistance to a group of antibiotics including G418. The cells were seeded in alpha minus MEM supplemented with 1 mg/ml G418. After 2 days, the cells were trypsinized and seeded in hybridoma cloning plates (Greiner, Germany) in alpha minus MEM supplemented with 10, 25, or 50 ng/ml of methotrexate plus 1 mg/ml G418. After about 10-14 days single clones were trypsinized and then seeded in 6-well petri dishes or 10 ml flasks using different concentrations of methotrexate (50 nM, 100 nM 200 nM, 400 nM, 800 nM). Clones growing at the highest concentrations of methotrexate were then transferred to new 6-well plates containing even higher concentrations of methotrexate (1 μM, 2 μM, 5 μM, 10 mM, 20 mM). The same procedure was repeated until clones were obtained which grow at a concentration of 100-200 μM. Expression of the desired gene product was analyzed by SDS-PAGE and Western blot or by reverse phase HPLC analysis.

### Example 4

### Tissue distribution of CESP Protein Expression

Northern blot analysis was carried out to examine CESP gene expression in human tissues, using the methods described by Sambrook et al., cited above. A cDNA probe containing the entire nucleotide sequence of the CESP protein (SEQ ID NO: 1) was labeled with ³²P using the rediprime™ DNA labeling system (Amersham Life Science), according to manufacturer's instructions. After labelling, the probe was purified using a CHROMA SPIN-100™ column (Clontech Laboratories, Inc.), according to manufacturer's protocol number PT1200-1. The purified labelled probe was then used to examine various human tissues for CESP mRNA.

Multiple Tissue Northern (MTN) blots from human heart, brain, placenta, lung, liver, skeletal muscle, kidney, pancreas, spleen, thymus, prostate, testis, ovary, small intestine, colon, and peripheral blood leukocytes were obtained from Clontech and were examined with labelled probe using ExpressHyb™ hybridization solution (Clontech) according to manufacturer's protocol number PT1190-1. Following hybridization and washing, the blots were mounted and exposed to film at −70° C. overnight, and films developed according to standard procedures. An abundant 2.6 kilobase transcript was detected in heart and brain. A weaker 2.6 kilobase signal was detected in endothelial cells, amniotic cells, smooth muscle, HSC172 cells and osteoblastoma cells.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the amino acid sequence at position about −21 to about 329 in SEQ ID NO:2;
(b) a nucleotide sequence encoding the amino acid sequence at position about −20 to about 329 in SEQ ID NO:2;
(c) a nucleotide sequence encoding the amino acid sequence at position about 1 to about 329 in SEQ ID NO:2;
(d) a nucleotide sequence encoding the CESP polypeptide having the complete, amino acid sequence encoded by the cDNA clone contained in ATCC Deposit No. 97728;
(e) a nucleotide sequence encoding the mature CESP polypeptide having the amino acid sequence encoded by the cDNA clone contained in ATCC Deposit No. 97728; and
(f) a nucleotide sequence complementary to any of the nucleotide sequences in (a), (b), (c), (d), or (e).

2. The nucleic acid molecule of claim 1 wherein said polynucleotide has the complete nucleotide sequence in SEQ ID NO:1.

3. The nucleic acid molecule of claim 1 wherein said polynucleotide has the nucleotide sequence in SEQ ID NO:1 encoding the CESP polypeptide having the complete amino acid sequence in SEQ ID NO:2.

4. The nucleic acid molecule of claim 1 wherein said polynucleotide has the nucleotide sequence in SEQ ID NO:1 encoding the mature CESP polypeptide having the amino acid sequence in SEQ ID NO:2.

5. The nucleic acid molecule of claim 1 wherein said polynucleotide has the complete nucleotide sequence of the cDNA clone contained in ATCC Deposit No. 97728.

6. The nucleic acid molecule of claim 1 wherein said polynucleotide has the nucleotide sequence encoding the CESP polypeptide having the complete amino acid sequence encoded by the cDNA clone contained in ATCC Deposit No. 97728.

7. The nucleic acid molecule of claim 1 wherein said polynucleotide has the nucleotide sequence encoding the mature CESP polypeptide having the amino acid sequence encoded by the cDNA clone contained in ATCC Deposit No. 97728.

8. An isolated nucleic acid molecule comprising a polynucleotide which hybridizes under stringent hybridization conditions to a polynucleotide having a nucleotide sequence identical to a nucleotide sequence in (a), (b), (c), (d), or (e) of claim 1 wherein said polynucleotide which hybridizes does not hybridize under stringent hybridization conditions to a polynucleotide having a nucleotide sequence consisting of only A residues or of only T residues.

9. An isolated nucleic acid molecule comprising a polynucleotide which encodes the amino acid sequence of an epitope-bearing portion of a CESP polypeptide having an amino acid sequence in (a), (b), (c), (d), or (e) of claim 1.

10. The isolated nucleic acid molecule of claim 9, which encodes an epitope-bearing portion of a CESP polypeptide selected from the group consisting of: a polypeptide comprising amino acid residues from about amino acid −1 to about 65 in SEQ ID NO:2; a polypeptide comprising amino acid residues from about 71 to about 105 in SEQ ID NO:2; a polypeptide comprising amino acid residues from about 114 to about 136 in SEQ ID NO:2; a polypeptide comprising amino acid residues from about 148 to about 169 in SEQ ID NO:2; a polypeptide comprising amino acid residues from about 174 to about 198 in SEQ ID NO:2; a polypeptide comprising amino acid residues from about 213 to about 229 in SEQ ID NO:2; a polypeptide comprising amino acid residues from about 234 to about 253 in SEQ ID NO:2; and a polypeptide comprising amino acid residues from about 267 to about 315 in SEQ ID NO:2.

11. An isolated nucleic acid molecule, comprising a polynucleotide having a sequence selected from the group consisting of:
(a) the nucleotide sequence of a fragment, wherein said fragment comprises at least 50 contiguous nucleotides of the coding region of the sequence shown in SEQ ID NO: 1, provided that said isolated nucleic acid molecule is not SEQ ID NO:12, SEQ ID NO:13, SEQ ID NO:14, SEQ ID NO:15, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or SEQ ID NO:36, or any subfragment thereof; and
(b) a nucleotide sequence complementary to a nucleotide sequence in (a).

12. A method for making a recombinant vector comprising inserting an isolated nucleic acid molecule of claim 1 into a vector.

13. A recombinant vector produced by the method of claim 12.

14. A method of making a recombinant host cell comprising introducing the recombinant vector of claim 13 into a host cell.

15. A recombinant host cell produced by the method of claim 14.

16. A recombinant method for producing a CESP polypeptide, comprising culturing the recombinant host cell of claim 15 under conditions such that said polypeptide is expressed and recovering said polypeptide.

17. An isolated CESP polypeptide having an amino acid sequence at least 95% identical to a sequence selected from the group consisting of:
(a) amino acids about −21 to about 329 in SEQ ID NO:2;
(b) amino acids about −20 to about 329 in SEQ ID NO:2;
(c) amino acids about 1 to about 329 in SEQ ID NO:2;
(d) the amino acid sequence of the CESP polypeptide having the complete amino acid sequence encoded by the cDNA clone contained in ATCC Deposit No. 97728; and
(e) the amino acid sequence of the mature CESP polypeptide having the amino acid sequence encoded by the cDNA clone contained in ATCC Deposit No. 97728; and
(f) the amino acid sequence of an epitope-bearing portion of any one of the polypeptides of (a), (b), (c), (d), or (e).

18. An isolated polypeptide comprising an epitope-bearing portion of the CESP protein, wherein said portion is selected from the group consisting of: a polypeptide comprising amino acid residues from about amino acid −1 to about 65 in SEQ ID NO:2; a polypeptide comprising amino acid residues from about 71 to about 105 in SEQ ID NO:2; a polypeptide comprising amino acid residues from about 114 to about 136 in SEQ ID NO:2; a polypeptide comprising amino acid residues from about 148 to about 169 in SEQ ID NO:2; a polypeptide comprising amino acid residues from about 174 to about 198 in SEQ ID NO:2; a polypeptide comprising amino acid residues from about 213 to about 229 in SEQ ID NO:2; a polypeptide comprising amino acid residues from about 234 to about 253 in SEQ ID NO:2; and a polypeptide comprising amino acid residues from about 267 to about 315 in SEQ ID NO:2.

19. An isolated nucleic acid molecule comprising a polynucleotide encoding a CESP polypeptide wherein, except for one to fifty conservative amino acid substitutions, said polypeptide has a sequence selected from the group consisting of:
(a) a nucleotide sequence encoding the amino acid sequence at position about −21 to about 329 in SEQ ID NO:2;
(b) a nucleotide sequence encoding the amino acid sequence at position about −20 to about 329 in SEQ ID NO:2;
(c) a nucleotide sequence encoding the amino acid sequence at position about 1 to about 329 in SEQ ID NO:2;
(d) a nucleotide sequence encoding the CESP polypeptide having the complete amino acid sequence encoded by the cDNA clone contained in ATCC Deposit No. 97728;
(e) a nucleotide sequence encoding the mature CESP polypeptide having the amino acid sequence encoded by the cDNA clone contained in ATCC Deposit No. 97728; and
(f) a nucleotide sequence complementary to any of the nucleotide sequences in (a), (b), (c), (d), or (e).

20. An isolated CESP polypeptide wherein, except for one to fifty conservative amino acid substitutions, said polypeptide has a sequence selected from the group consisting of:
(a) amino acids about −21 to about 329 in SEQ ID NO:2;
(b) amino acids about −20 to about 329 in SEQ ID NO:2;
(c) amino acids about 1 to about 329 in SEQ ID NO:2;
(d) the amino acid sequence of the CESP polypeptide having the complete amino acid sequence encoded by the cDNA clone contained in ATCC Deposit No. 97728; and
(e) the amino acid sequence of the mature CESP polypeptide having the amino acid sequence encoded by the cDNA clone contained in ATCC Deposit No. 97728; and
(f) the amino acid sequence of an epitope-bearing portion of any one of the polypeptides of (a), (b), (c), (d), or (e).
